SEQUENCE LISTING (2 sequences)

SEQ ID NO: 1 - protein, 151 aa, Homo sapiens
MERPEPELIRQSWRAVSRSPLEHGTVLFARLFALEPDLLPLFQYNCRQFSSPEDCLSSPEFLDHIRKVMLVIDAAVTNVEDLSSLEEYLASLGRKHRAVGVKLSSFSTVGESLLYMLEKCLGPAFTPATRAAWSQLYGAVVQAMSRGWDGE

SEQ ID NO: 2 - DNA, 1885 bp, Homo sapiens
  source 1..1885: mol_type = genomic DNA
ttcccaggccaccatagcggctggcggagggagcgcgcgccttgctggcctggagggggcgggggccgtggcggctttaaagcgcccagcccaggcgtcgcggggtggggcggctctggcggctgcggggcgcagggcgcagcggccaagcggggtccccggaagcacagctggggtgtctccacctacgactggccgcgcgccttttctctcccgcgccagggaaggagcggctgcggcccccgccgggcggaggcacggggggcgtacgaggggcggaggggaccgcgtcgcggaggagatggcgcggcacgtgcggtgacggcacccgagccctgagggtcccagccccgcgctccgcgtccccgggacagcatggagcgcccggagcccgagctgatccggcagagctggcgggcagtgagccgcagcccgctggagcacggcaccgtcctgtttgccaggctgtttgccctggagcctgacctgctgcccctcttccagtacaactgccgccagttctccagcccagaggactgtctctcctcgcctgagttcctggaccacatcaggaaggtgatgctcgtgattgatgctgcagtgaccaatgtggaagacctgtcctcactggaggagtaccttgccagcctgggcaggaagcaccgggcagtgggtgtgaagctcagctccttctcgacagtgggtgagtctctgctctacatgctggagaagtgtctgggccctgccttcacaccagccacacgggctgcctggagccaactctacggggccgtagtgcaggccatgagtcgaggctgggatggcgagtaagaggcgaccccgcccggcagcccccatccatctgtgtctgtctgttggcctgtatctgttgtagcccaggctccccaagcttccctgcatcttggtccttgtccccttggccacactggagaggtgatggggcagggctgggtctcagtatcctagagtccagctgcagaaggagtggcttttcctccaggaaggggcttctgggtgtcccctcatccccagtagcctctttcttgcgtttctttttaccttttttggcactccctctgaccccgcgatgagtgttttggtggcagaggtgggatgagctggaaaggtatggaggtgggagaggatggggctcttctgtctgtcctgcttcttcaggtgagtgcaggccaaggcgggggtgagatggctgagcttccagcgccttctgtcctgcctgcccagtcccttcactgctttcctgccccaagatggcttgcttttcacaaataaagagaaagagcagctttagccttcttggtggaatcccaggcagtgggagcagaatcagaactgccagggaagggaagggggacctgggtctcaatgggtctcatttgagtctcgcgggctgtgcagatgccctgacagagtcggtttcctttggcggcattccctttccctcattcagcacttctgctgggaactccctgactattccgctgctgcaggaacccagctagctggccaggtggggaggggctggggaccggccaggaaggaggggtgacttcatcccagagagacccgagttcccccagcccttcatcaccaacccgctcctgcaggagtgagtcttacctcccctggccctcctttctggctcagcctgcagcgactgtgaggccacagctcctcagattcactgcccgctgtgtgccagtactcaggcagctggagagaagagaaggcagcagcagaggcccccgccctcaccccagccatctgcacttgtaccatttgctctgtgctgactgtggtcctataaattcatgagaaataaactggttctgtgtgcaaaaaaaaa

(19) United States
(12) Patent Application Publication
(10) Pub. No.: US 2026/0325611 A1
(43) Pub. Date: Oct. 22, 2026

(54) INTRAVENOUS ADMINISTRATION OF NEUROGLOBIN FOR TREATING NEUROLOGICAL DISORDERS

(71) Applicants: INSERM (INSTITUT NATIONAL DE LA SANTÉ ET DE LA RECHERCHE MÉDICALE), PARIS (FR); ASSOCIATION FRANCAISE CONTRE LES MYOPATHIES (A.F.M), PARIS (FR); UNIVERSITE PARIS CITE, Paris (FR)

(72) Inventors: Marisol CORRAL-DEBRINSKI, PARIS (FR); Pierre GRESSENS, PARIS (FR)

(21) Appl. No.: 18/881,057

(22) PCT Filed: Jul. 4, 2023
(86) PCT No.: PCT/EP2023/068369

§ 371 Date: Jan. 3, 2025

(30) Foreign Application Priority Data
Jul. 5, 2022 (EP) 22306002.1

(51) Int. Cl. A61K 38/41; A61K 48/00; C12N 15/86

(52) U.S. Cl. CPC A61K 38/41; A61K 48/0033; A61K 48/005; A61K 48/0075; C12N 15/86; C12N 2750/14143

(57) ABSTRACT

Using Harlequin (Hq) mice (a model developing an ataxic phenotype), the present inventors have confirmed that neuroglobin-based gene therapy represents a very promising tool for the treatment of neurological diseases. Hq mice were subjected to either intravenous (injection in the vein of the retro-orbital sinus) or local (neurosurgery in cerebellar hemispheres) administration of a neuroglobin-encoding AAV vector. The present inventors have demonstrated that in comparison to other modes of administration, the very specific intravenous administration of neuroglobin allows significantly increasing the survival rate of treated Hq mice, minimizing their weight loss, preventing cerebellar tissue degeneration, and preserving Purkinje cells and their dendritic arborizations. The present inventors have thus shown that the very specific intravenous administration of neuroglobin provides strong neuroprotective effects and thereby represents an extremely promising therapeutic strategy for treating neurological disorders. Accordingly, the present invention pertains to neuroglobin for use in the treatment of neurological disorders, wherein neuroglobin is administered intravenously to a patient in need thereof.

### TECHNICAL FIELD

The present invention pertains to the specific use of neuroglobin administered intravenously for the treatment neurological disorders.

### BACKGROUND ART

The functional integrity of the central nervous system relies on complex mechanisms in which the mitochondria are crucial players because of their involvement in a multitude of bioenergetics and biosynthetic pathways (Thompson et al. J Inherit Metab Dis. 2020; 43 (1): 36-50). Mitochondrial diseases are among the most prevalent groups of inherited neurological disorders, affecting up to 1 in 5000 adults (Gorman et al. Ann Neurol. 2015; 77 (5): 753-759). Despite the remarkable progress that has been achieved in the last 34 years in the discovery of their genetic causes, therapies which effectively improve patient's quality of life are still unavailable (Thompson et al. 2020; Bottani et al. Pharmaceutics. 2021; 12 (11)).

Gene therapy represents one promising strategy to treat devastating conditions due to neuronal cell death. Indeed, encouraging results have been recently reported for Spinal Muscular Atrophy (Aslesh et al. Cells. 2022; 11 (3)) and for Leber Hereditary Optic Neuropathy (Zhang et al. Curr Gene Ther. 2019; 19 (2): 134-138; Biousse et al. J Neuroophthalmol. 2021; 41 (3): 309-315). However, this requires identifying appropriate gene candidates whose expressions are impaired in neurological disorders and whose administration effectively allows alleviating/reversing neuronal cell death.

Neuroglobin (encoded by the Ngb gene) was identified in 2000 as a member of the globin superfamily (Burmester et al. Nature. 2000; 407 (6803): 520-523). The protein of 151 amino acids is highly abundant in the brain, being present in both neurons (Hundahl et al. Brain Res. 2010; 1331:58-73) and astrocytes (Chen et al. Neurosci Lett. 2015; 606:194-199). The neuroprotective role of neuroglobin has been largely documented in vitro and in vivo (Ascenzi et al. Mol Aspects Med. 2016; 52:1-48; Van Acker et al. Mol Neurobiol. 2019; 56 (3): 2101-2122). It is now well accepted that the majority of neuroglobin localizes to the mitochondria where the protein ensures the functional integrity of the respiratory chain (Lechauve et al. Biochim Biophys Acta. 2012; 1823 (12): 2261-2273; Cwerman-Thibault et al. Neurobiol Dis. 2021; 159:105483).

In view of these properties, neuroglobin qualifies as a very interesting candidate for gene therapy. The use of neuroglobin agonists for the prevention/treatment of mitochondrial RCCI and/or RCCIII deficiency diseases has already proven successful (see international patent application published under reference WO2015/044462).

However, there is always a need for the development of new and more efficient treatments for neurological disorders.

### SUMMARY

The invention is defined by the claims.

Using Harlequin (Hq) mice (a model developing an ataxic phenotype), the present inventors have confirmed that neuroglobin-based gene therapy represents a very promising tool for the treatment of neurological diseases. Furthermore, and more interestingly, the present inventors have demonstrated that in comparison to other modes of administration, the very specific intravenous administration of neuroglobin allows significantly increasing the survival rate of treated Hq mice, minimizing their weight loss, preventing cerebellar tissue degeneration, and preserving Purkinje cells and their dendritic arborizations.

The present inventors have thus shown that the very specific intravenous administration of neuroglobin provides strong neuroprotective effects and thereby represents an extremely promising therapeutic strategy for treating neurological disorders.

Accordingly, the present invention pertains to neuroglobin for use in the treatment of neurological disorders, wherein neuroglobin is administered intravenously to a patient in need thereof.

According to a specific embodiment, the neurological disorder is associated with a mitochondrial disease, more particularly a mitochondrial disease associated with respiratory chain complex I (RCCI) deficiency and/or respiratory chain complex III (RCCIII) deficiency.

In the context of the present invention, the neurological disorder to be treated is preferably ataxia, more specifically hereditary ataxia such as Friedreich ataxia, cerebellar ataxia or spinocerebellar ataxia.

### BRIEF DESCRIPTION OF DRAWINGS

FIG. 1 Physical maps of the AAV2/9-Aifm1, AAV2/9-Ngb and AAV2/9-GFP vectors: The mouse Aifm1 sequence corresponds to the Mus musculus apoptosis-inducing factor, mitochondrion-associated 1 (Aifm1), transcript variant 1, mRNA (NM_012019.3). The mouse Ngb sequence corresponds to the Mus musculus neuroglobin transcript variant 2 mRNA (NM_022414.2) and was retrieved from the NCBI website. Sequences are inserted into the pAAV-IRES-hrGFP plasmid (Agilent Technologies), both coding sequences (CDS) are in frame with three FLAG epitopes and transcribed under the control of the Cytomegalovirus promoter and the β-globin intron. The construction contains the UTRs at the 5′ and the 3′ extremities of each mRNA. Additionally, each plasmid possesses a cassette allowing the expression of the recombinant humanized green fluorescent protein (hrGFP) translated from the encephalomyocarditis virus internal ribosome entry site. In the bottom of the figure, it is illustrated the schematic representation of the empty vector, which does not have any transgene inserted in the MCS. It was used as a negative control in our experience.

### DESCRIPTION OF EMBODIMENTS

The present inventors have submitted Harlequin (Hq) mice to neuroglobin gene therapy via different routes of administration and have shown that intravenous neuroglobin administration, specifically, allows obtaining effective neuroprotective effects.

Hq mice develop vision loss and cerebellar ataxia as they age (Klein et al. Nature. 2002; 419 (6905): 367-374). They display a proviral insertion in the first intron of the Apoptosis-inducing factor gene (Aifm1), resulting in the almost complete depletion of the corresponding protein, Apotosis-Inducing Factor-Aif (Klein et al. 2002). Therefore, Hq mice display progressive degeneration of the retina, optic nerve, cerebellum, and cortical regions leading to irremediable blindness and ataxia as they age. The phenotype is due to a severe respiratory chain complex I deficit which results in neuronal cell degeneration (Cwerman-Thibault et al. Neurobiol Dis. 2021; 159:105483; Vahsen et al. Embo J. 2004; 23 (23): 4679-4689; Bouaita et al. Brain. 2012; 135 (Pt 1): 35-52; Lechauve C, Augustin et al. Mol Ther. 2014; 22 (6): 1096-1109).

Based on this model, the inventors have shown that:
the intravenous administration of a neuroglobin-encoding vector does not result in deleterious effects on body weight of Harlequin mice which, in normal conditions, exhibit growth retardation. Treated Harlequin mice show a noticeable increase of body weight indicating that their overall health status is not deteriorated by neuroglobin overexpression;
the weight, the surface and the overall morphology of the cerebellum in Hq mice is better preserved after intravenous administration of the neuroglobin-encoding vector as compared to an administration inside the tissue (cerebellar hemispheres) by neurosurgery;
the number of Purkinje cells and their density are higher when the neuroglobin-encoding vector was administered intravenously as compared to an administration by stereotactic neurosurgery in cerebellar hemispheres (Purkinje cells degeneration is one of the most common hallmarks of hereditary ataxias); and that
Harlequin mice treated with a neuroglobin-encoding vector by intravenous administration exhibit both an enhanced locomotor capability and a stronger spatial memory as compared to mice treated with a local administration of the vector.

The present application therefore shows that a specific intravenous administration of neuroglobin provides multiple unexpected and surprising effects as compared to other routes of administration. The present application therefore shows that specifically administering neuroglobin intravenously allows efficiently preventing/reversing neurodegenerative processes observed in neurological disorders.

Accordingly, the present invention pertains to neuroglobin for use in the treatment of neurological disorders, wherein neuroglobin is administered intravenously.

Neuroglobin or “NGB” is an oxygen-binding protein that is related to members of the globin family. Neuroglobin is encoded by the Ngb gene which is highly conserved among other vertebrates. It is expressed in the central and peripheral nervous system where it is involved in increasing oxygen availability and providing protection under hypoxic/ischemic conditions. Human neuroglobin has the amino acid sequence: MERPEPELIRQSWRAVSRSPLEHGTVLFARLFALEPDLLPLFQYNCRQFSSPEDCLSSPEFLDHIR KVMLVIDAAVTNVEDLSSLEEYLASLGRKHRAVGVKLSSFSTVGESLLYMLEKCLGPAFTPATRAA WSQLYGAVVQAMSRGWDGE (SEQ ID No. 1)—accessible under reference Q9NPG2 in the Uniprot database) and is encoded by the human Ngb gene which has the nucleic acid sequence: ttcccaggccaccatagcggctggcggagggagcgcgcgccttgctggcctggagggggcgggggccgtggcggctttaaagcgccc agcccaggcgtcgcggggggggcggctctggcggctgcggggcgcagggcgcagcggccaagcggggtccccggaagcacagct ggggtgtctccacctacgactggccgcgcgccttttctctcccgcgccagggaaggagcggctgcggcccccgccgggggaggcacg gggggcgtacgaggggcggaggggaccgcgtcgcggaggagatggcgcggcacgtgcggtgacggcacccgagccctgagggtc ccagccccgcgctccgcgtccccgggacagcatggagcgcccggagcccgagctgatccggcagagctggcgggcagtgag ccgcagcccgctggagcacggcaccgtcctgtttgccaggctgtttgccctggagcctgacctgctgcccctcttccagtacaa ctgccgccagttctccagcccagaggactgtctctcctcgcctgagttcctggaccacatcaggaaggtgatgctcgtgattgat gctgcagtgaccaatgtggaagacctgtcctcactggaggagtaccttgccagcctgggcaggaagcaccgggcagtgggt gtgaagctcagctccttctcgacagtgggtgagtctctgctctacatgctggagaagtgtctgggccctgccttcacaccagcca cacgggctgcctggagccaactctacggggccgtagtgcaggccatgagtcgaggctgggatggcgagtaagaggcgaccc cgcccggcagcccccatccatctgtgtctgtctgttggcctgtatctgttgtagcccaggctccccaagcttccctgcatcttggtccttgtcccct tggccacactggagaggtgatggggcagggctgggtctcagtatcctagagtccagctgcagaaggagtggcttttcctccaggaaggg gcttctgggtgtcccctcatccccagtagcctctttcttgcgtttctttttaccttttttggcactccctctgaccccgcgatgagtgttttggtggcaga ggtgggatgagctggaaaggtatggaggtgggagaggatggggctcttctgtctgtcctgcttcttcaggtgagtgcaggccaaggcggg ggtgagatggctgagcttccagcgccttctgtcctgcctgcccagtcccttcactgctttcctgccccaagatggcttgcttttcacaaataaag agaaagagcagctttagccttcttggtggaatcccaggcagtgggagcagaatcagaactgccagggaagggaagggggacctgggt ctcaatgggtctcatttgagtctcgcgggctgtgcagatgccctgacagagtcggtttcctttggcggcattccctttccctcattcagcacttctg ctgggaactccctgactattccgctgctgcaggaacccagctagctggccaggtggggaggggctggggaccggccaggaaggaggg gtgacttcatcccagagagacccgagttcccccagcccttcatcaccaacccgctcctgcaggagtgagtcttacctcccctggccctccttt ctggctcagcctgcagcgactgtgaggccacagctcctcagattcactgcccgctgtgtgccagtactcaggcagctggagagaagaga aggcagcagcagaggcccccgccctcaccccagccatctgcacttgtaccatttgctctgtgctgactgtggtcctataaattcatgagaaat aaactggttctgtgtgcaaaaaaaaa (SEQ ID No. 2). The region in bold corresponds to the coding region of the gene. The first 375 nucleotides correspond to the 5′UTR, and the last 1054 nucleotides correspond to the 3′UTR.

As explained above, neuroglobin has powerful neuroprotectant effects. It can therefore be advantageously used for treating disorders affecting neurons, in particular neurological disorders.

“Neurological disorders” encompasses a well-defined group of diseases that affect the nervous system. The world health organization defines neurological disorders as being diseases of the central and peripheral nervous system affecting the brain, spinal cord, cranial nerves, peripheral nerves, nerve roots, autonomic nervous system, neuromuscular junction, and muscles. These disorders include epilepsy, Alzheimer disease and other dementias, cerebrovascular diseases including stroke, migraine and other headache disorders, multiple sclerosis, Parkinson's disease, ataxias, neuroinfections, brain tumors and traumatic disorders of the nervous system due to head trauma.

As thoroughly explained in the international application published under reference WO2015/044462, neuroglobin can efficiently be used in the treatment or prevention of mitochondrial diseases associated with respiratory chain complex I (RCCI) deficiency and/or respiratory chain complex III (RCCIII) deficiency.

Therefore, according to a specific embodiment the present invention pertains to the intravenous administration of neuroglobin for treating neurological disorders associated with mitochondrial diseases, in particular with mitochondrial disorders associated with respiratory chain complex I (RCCI) deficiency and/or respiratory chain complex III (RCCIII) deficiency.

As used herein, the term “Mitochondrial disease” refers to disorders in which deficits in mitochondrial respiratory chain activity contribute to the development of pathophysiology of such disorders in a mammal. Mitochondrial disorders may be caused by acquired or inherited mutations in mitochondrial DNA (mtDNA) or in nuclear genes that code for mitochondrial components. They may also be the result of acquired mitochondrial dysfunction due to adverse effects of drugs, infections, or other (environmental . . . ) causes.

A “mitochondrial disease associated with respiratory chain complex I deficiency” or “a mitochondrial disease associated with RCCI deficiency” refers to a mitochondrial disease in which a dysregulation, a reduction or an abolition of RCCI complex activity is observed. The term “mitochondrial disease associated with RCCI deficiency” also refers to a mitochondrial disease induced by RCCI deficiency or in which RCCI deficiency increases the risk of developing such mitochondrial disease. Examples of mitochondrial diseases associated with RCCI deficiency may be Leber's hereditary optic neuropathy (LHON), MELAS (Mitochondrial encephalomyopathy, lactic acidosis, and stroke-like episodes), MERRF (Myoclonic Epilepsy with Ragged Red Fibers), Leigh Syndrome,LS (subacute necrotizing encephalomyelopathy is a progressive neurological disease defined by specific neuropathological features associating brainstem and basal ganglia lesions), Leukoencephalopathy (brain white matter disease), Cardiomiopathy, Hepatopathy with tubulopathy, and Fatal infantile multisystem disorder (for review see Scheffler J Inherit Metab Dis, 2014 DOI 10. 1007/s10545-014-9768-6; Papa and De Rasmo, Trends in Molecular Medicine, 2013, Vol. 19, No. 1:61-69 and the MITOMAP website).

As used herein, the term “respiratory chain complex I” or “RCCI” refers to a protein complex located in the mitochondrial inner membrane that forms part of the mitochondrial respiratory chain. RCCI contains about 45 different polypeptide subunits, including NADH dehydrogenase (ubiquinone), flavin mononucleotide and several different iron-sulfur clusters containing non-heme iron. The iron undergoes oxidation-reduction between Fe(II) and Fe(III), and catalyzes proton translocation linked to the oxidation of NADH by ubiquinone. RCCI is also named NADH: quinone oxidoreductase (E.C. 1.6.5.3). RCCI function or RCCI activity may be measured by: (1) a very accurate and powerful spectrophotometric assay designed for minuscule biological samples (Benit et al., Clinica Chimica Acta 374 (2006) 81-86); (2) the biochemical analysis of respiratory chain (oxidative phosphorylation) complexes using Blue native (BN) polyacrylamide gel electrophoresis (PAGE) after the extraction from tissues or cells of enriched mitochondrial membranes; both the in-gel activity of respiratory chain complexes and the protein composition of each one of them could be analyzed (Calvaruso et al., Methods 46 (2008) 280-286).

As used herein, the term “a mitochondrial disease associated with respiratory chain complex III deficiency” or “a mitochondrial disease associated with RCCIII deficiency” refers to a mitochondrial disease in which a dysregulation, a reduction or an abolition of RCCIII complex activity is observed. The term “mitochondrial disease associated with RCCIII deficiency” also refers to a mitochondrial disease induced by RCCIII deficiency or in which RCCIII deficiency increases the risk of developing such mitochondrial disease. Examples of mitochondrial diseases associated with RCCIII deficiency may be Encephalopathy, Hepatic failure and tubulopathy, Leigh Syndrome, GRACILE and GRACILE-like syndromes (growth retardation, aminoaciduria, cholestasis, iron overload, lactic acidosis and early death), Bjornstad Syndrome (sensorineural hearing loss and twisted hairs), Hypoglycemia, Lactic acidosis, LHON, progressive exercise intolerance, degeneration of cerebellar neurons and progressive psychiatric syndrome (for review see Benit et al., Biochimica et Biophysica Acta 1793 (2009) 181-185; http://www.mitomap.org/MITOMAP).

As used herein, the term “respiratory chain complex III” or “RCCIII” refers to a protein complex located in the mitochondrial inner membrane that forms part of the mitochondrial respiratory chain. RCCIII contains about 11 polypeptide subunits including four redox centers: cytochrome b/b6, cytochrome c1 and a 2Fe-2S cluster. RCCIII function is to catalyze the oxidation of ubiquinol by oxidized cytochrome c1. RCCIII is also named bc1 complex; ubiquinol cytochrome c reductase (EC 1.10.2.2). The RCCIII function or RCCIII activity may be measured by: (1) a very accurate and powerful spectrophotometric assay designed for minuscule biological samples (Benit et al., Clinica Chimica Acta 374 (2006) 81-86); (2) the biochemical analysis of respiratory chain (oxidative phosphorylation) complexes using Blue native (BN) polyacrylamide gel electrophoresis (PAGE) after the extraction from tissues or cells of enriched mitochondrial membranes; both the in-gel activity of respiratory chain complexes and the protein composition of each one of them could be analyzed (Calvaruso et al., Methods 46 (2008) 280-286).

Intravenous administration of neuroglobin prevents neuronal degradation. As such, it can be used in the treatment of all neurological disorders, and particularly for the treatment of disorders involving neurodegeneration, i.e. neurodegenerative disorder. Neurodegenerative disorders include Alzheimer's disease, ataxia, Huntington's disease, Parkinson's disease, motor neuron disease, leukodystrophies or multiple system atrophy. Contrary to acute neurological disorders such a stroke, neurodegenerative disorders are evolutive disorders that induce prolonged and progressive neuronal damages. They progress over several years and involve specific mechanisms leading, over the course of their evolution, to neural cell death and permanent brain damage.

According to a preferred embodiment, the present invention pertains to the administration of intravenous neuroglobin for treating ataxia, more specifically hereditary ataxia such as Friedreich ataxia, cerebellar ataxia or spinocerebellar ataxia.

“Ataxias” refers to a group of neurological disorders which affect coordination, balance and speech. They usually result from a damage in the cerebellum. Ataxias may cause difficulty with walking and balance, hand coordination, speech and swallowing, and eye movements.

“Hereditary ataxias” are inherited, in opposition to a related group of neurological disorders that are acquired through accidents, injuries, or other external agents. Hereditary ataxias are characterized by degenerative processes in the brain and spinal cord that leads to gait abnormalities accompanied often by poor eye-hand coordination and dysarthria (see for review Perlman S or Bird T. “Hereditary ataxia overview.” GeneReviews@ [internet] (2019)). Hereditary ataxias include e.g. Friedreich ataxia, cerebellar ataxia or spinocerebellar ataxia (an autosomal dominant ataxias).

In the context of the present invention, neuroglobin is specifically administered intravenously. The skilled person is familiar with several techniques for intravenously administering a neuroglobin in the context of the present invention. Techniques useful in the context of the present invention are e.g. used in the clinical trials referenced under the following ClinicalTrials identifiers: NCT02122952 NCT03461289, NCT03952637, NCT03362502, NCT05092685, NCT04998396, NCT03955679 or NCT04040049. The corresponding composition may be presented in unit dose form in ampoules, pre-filled syringes small volume infusion or in multi-dose containers with an added preservative. The compositions may be in the form of suspensions, solutions, or emulsions in oily or aqueous vehicles, for example solutions in aqueous polyethylene glycol. Examples of oily or non-aqueous carriers, diluents solvents or vehicles include propylene glycol, polyethylene glycol, vegetable oils (e.g., olive oil, and injectable organic esters (e.g., ethyl oleate), and may contain formulation agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water.

In the context of the present invention, neuroglobin can be administered as a polypeptide, by administering the mature NGB protein. However, neuroglobin is preferably administered as a gene therapy. Gene therapy may be carried out by means of supplementation of target cells with a functional neuroglobin. Production of a suitable gene product may be achieved by using recombinant techniques. For example, a suitable vector may be inserted into a host cell and expressed in that cell. Gene therapy is particularly advantageous for the treatment of disorders such as neurodegenerative disorders as it allows for the prolonged and sustained release of the target gene in the target cells. Gene therapy is typically achieved by administering a polynucleotide encoding the target gene to the patient.

Accordingly, the present invention pertains to a neuroglobin-encoding polynucleotide for use in the treatment of neurological disorders. A method for treating a neurological disorder comprising administering, to a patient in need thereof, a neuroglobin-encoding polynucleotide is therefore also disclosed herein. Said neuroglobin-encoding polynucleotide is administered in a therapeutically effective amount.

In the context of the present invention, the “patient” or “subject” is a mammal (e.g. a dog, a cat, a pig, a rodent or a primate). In a particular embodiment, the patient is a human. Typically, said polynucleotide is comprised in an expression cassette.

An “expression cassette” refers to a linear or circular nucleic acid molecule. This expression cassette also refers to DNA and RNA sequences which allow for the production of a functional nucleotide sequence in a suitable host cell. Typically, the expression cassette comprises a polynucleotide encoding neuroglobin operatively linked to at least one transcriptional regulatory sequence. Typically, the expression cassette comprises a polynucleotide encoding neuroglobin protein, said polynucleotide being operatively linked to at least one transcriptional regulatory sequence for the expression of neuroglobin protein in target cells, said at least one transcriptional regulatory sequence being 3′UTR and/or 5′UTR NGB sequences. Typically, when the patient is human, said polynucleotide comprises the coding region of the human Ngb gene as shown in SEQ

ID No. 2. However, as would be understood by the skilled person, this polynucleotide can also be modified for optimizing its expression or the activity of its transcription product. Such optimized sequence variants can therefore also be used in the context of the present invention. The corresponding mRNA sequences can also be used in the context of the present invention.

The expression cassette can also include sequences required for proper translation of the nucleotide sequence of interest. The expression cassette may additionally contain selection marker genes. Typically, the cassette comprises in the 5′-3′ direction of transcription, a transcriptional and translation initiation region, a polynucleotide encoding the NGB protein, a transcription and translation termination region functional in mammalian cells.

The expression cassette may also include a multiple cloning site. In addition to the components mentioned above, the expression cassette of the present invention may comprise the components required for homologous recombination.

The term “operatively linked to” refers to the functional relationship of a nucleic acid with another nucleic acid sequence. Promoters, enhancers, transcriptional and translational stop sites, and other signal sequences are examples of nucleic acid sequences operatively linked to other sequences. For example, operative linkage of DNA to a transcriptional control element refers to the physical and functional relationship between the DNA and promoter such that the transcription of such DNA is initiated from the promoter by an RNA polymerase that specifically recognizes, binds to and transcribes the DNA.

As used herein, the term “transcriptional regulatory sequence”, “transcription regulatory sequence” or “regulatory sequences” refers to nucleotide sequences influencing the transcription, RNA processing or stability, or translation of the associated (or functionally linked) nucleotide sequence to be transcribed. The transcriptional regulatory sequence may have various localizations with the respect to the nucleotide sequences to be transcribed. The transcriptional regulatory sequence may be located upstream (5′ non-coding sequences), within, or downstream (3′ non-coding sequences) of the sequence to be transcribed (e.g., polynucleotide encoding NGB protein). The transcription regulating nucleotide sequences may be selected from the group consisting of enhancers, promoters, translation leader sequences, introns, 5′-untranslated sequences (5′UTR), 3′-untranslated sequences (3′UTR), and polyadenylation signal sequences. They include natural and synthetic sequences as well as sequences, which may be a combination of synthetic and natural sequences. As is noted above, the term “transcriptional regulatory sequence” is not limited to promoters. However, transcriptional regulatory sequence of the invention may comprise at least one promoter sequence (e.g., a sequence localized upstream of the transcription start of a gene capable to induce transcription of the downstream sequences), and/or at least one 3′UTR and/or one 5′UTR. According to a specific embodiment, the transcription regulating nucleotide sequence of the invention comprises the promoter sequence of the Ngb gene and/or the native 3′UTR of Ngb gene and/or native 5′UTR of Ngb gene. Furthermore, a fragment of the Ngb 3′UTR and/or of the Ngb 5′UTR may also be employed. According to another embodiment, the promoter is the promoter of another gene, for instance a gene strongly expressed in the brain. Advantageously, the promoter of the PGK1 gene is used. The transcription regulatory sequence of the invention can therefore comprise the promoter sequence of the PGK1 gene and/or the native 3′UTR of Ngb gene and/or native 5′UTR of Ngb gene. The presence of the native 3′UTR and 5′UTR of Ngb gene advantageously guarantees mRNA stability, translation capacity and the efficient delivery of Ngb inside the mitochondria.

As used herein, the term “Promoter” or “promoter sequence” refers to a DNA sequence in a gene, usually upstream (5′) to its coding sequence, which controls the transcription of the coding sequence such as the polynucleotide encoding neuroglobin protein by providing the recognition for RNA polymerase and other factors required for proper transcription. For example, the promoter may be the Ngb promoter, a variant or a fragment thereof, preferably, the human Ngb promoter. Promoters may contain DNA sequences that are involved in the binding of protein factors which control the effectiveness of transcription initiation in response to physiological or developmental conditions. Typically, the Ngb promoter may contain two GC boxes which are bound by Sp1 and Sp3 factors. According to the invention, the promoter sequence may also contain enhancer elements. An “enhancer” is a DNA sequence which can stimulate promoter activity. It may be an innate element of the promoter or a heterologous element inserted to enhance the level and/or tissue-specificity of a promoter. Typically, the promoter sequence of the invention is a ubiquitous promoter, a tissue-specific promoter or an inducible promoter. “Ubiquitous Promoters” refers to promoters that always direct gene expression in all tissues. The ubiquitous promoter may be eukaryotic or viral promoters. In one embodiment, the promoter sequence is an eukaryotic promoter selected from the group consisting of the chicken β-actin promoter (CBA), the composite CAG promoter (consisting of the CMV immediate early enhancer and the chicken β-actin promoter) and the human phosphoglycerate kinase 1 (PGK) promoter. According to another embodiment, the promoter sequence is a viral promoter such as the human cytomegalovirus (CMV) promoter. “Tissue-specific promoters” are promoters that direct gene expression almost exclusively in specific tissues, such as retina specific promoter or central nervous system specific promoter. The promoter may be selected among RGC specific promoters. Typically, the promoter is an “inducible promoter”, i.e. a promoter that directs gene expression in response to an external stimulus, such as light, heat-shock and chemical.

The “untranslated region” or “UTR” refers to either of the two regions immediately adjacent to the coding sequence on a strand of mature mRNA. When it is found on the 5′ side, it is called the 5′ UTR (or 5′ untranslated region), or if it is found on the 3′ side, it is called the 3′ UTR (or trailer sequence). As used herein, “3′UTR neuroglobin sequence” refers to the sequence of the 3′UTR of the Ngb gene, such as for example, the human Ngb 3′UTR. The human 3′UTR of the Ngb gene is the 3′ extremity of sequence SEQ ID No. 2 and corresponds to the 1054 last nucleotides of SEQ ID No. 2. As used herein, the term “5′UTR neuroglobin sequence” refers to the sequence of the 5′UTR of the Ngb gene. The human 5′UTR of the Ngb gene is the 5′ extremity of sequence SEQ ID No. 2 and corresponds to the 375 first nucleotides of SEQ ID No. 2 According to a specific embodiment, the neuroglobin-encoding polynucleotide comprises the neuroglobin gene coding region and the neuroglobin gene 5′UTR and/or 3′UTR.

In one embodiment, the expression cassette is comprised in an expression vector. Accordingly, the present invention also pertains to a neuroglobin-encoding polynucleotide for use in the treatment of neurological disorders, wherein said polynucleotide is comprised in an expression vector.

The term “vector” refers to a nucleic acid sequence capable of transporting into a cell another nucleic acid to which the vector sequence has been linked. The term “expression vector” includes any vector containing a gene construct or an expression cassette in a form suitable for expression by a cell. The “expression vector” may be any recombinant vector capable of expression of a NGB protein or fragment thereof. More particularly, the expression vectors used can be derived from bacterial plasmids, transposons, yeast episomes, from insertion elements, from yeast chromosomal elements, from viruses such as an adeno-associated virus (AAV) vector, a lentivirus vector, a retrovirus vector, a replication competent adenovirus vector, a replication deficient adenovirus vector and a gutless adenovirus vector, a herpes virus vector, baculoviruses, blinked as SV40 virus, the vaccinia virus, fox pox viruses, pseudorabies viruses. AAV and lentivirus vectors have emerged as the vectors of choice for gene transfer to the central nervous system as they mediate efficient long-term gene expression with no apparent toxicity. Moreover, several clinical trials have shown that direct infusion of AAV2 vectors into brain parenchyma in humans is well tolerated (Bowers et al., Human Molecular Genetics, 2011, Vol. 20, Review Issue 1 R28-R41). The expression cassette can be inserted into the expression vector by methods well known in the art.

The expression vector may include reporter genes. Examples of reporter genes encode luciferase, (green/red) fluorescent protein and variants thereof, like eGFP (enhanced green fluorescent protein), hrGFP (humanized recombinant green fluorescent protein), RFP (red fluorescent protein, like DsRed or DsRed2), CFP (cyan fluorescent protein), BFP (blue fluorescent protein), YFP (yellow fluorescent protein), β-galactosidase or chloramphenicol acetyltransferase, and the like. These sequences are selected depending on the host cell implemented.

According to one embodiment of the invention, the expression vector is a viral vector. The viral vector of the invention may be derived from retroviruses, herpes simplex viruses, adenoviruses or AAVs.

According to a preferred embodiment, the expression vector is preferably an adeno-associated virus (AAV) vector, preferably an AAV2 vector.

In one embodiment, the expression vector of the invention is an AAV vector comprising respectively the 5′ inverted terminal repeat (ITR5′) and 3′ inverted terminal repeat (ITR3′) sequences of the AAV, at the 5′ and 3′ ends of the expression cassette.

The expression “terminal inverted repeat sequence” or “ITR” means the terminal inverted repeat sequences of palindromic 145 base-pairs (bp) flanked at the 5 ‘and 3’ AAV vector. The ITRs sequences are essential for the integration, replication and packaging of the viral vector. AAV ITR's can be modified using standard molecular biology techniques. Accordingly, AAV ITRs used in the vectors of the invention need not have a wild-type nucleotide sequence, and may be altered, e.g., by the insertion, deletion or substitution of nucleotides. Indeed, the ITR5 ‘and ITR3’ are not necessarily identical but are functional. “Functional ITR sequences” means ITR sequences that allow for the vector replication and packaging. Additionally, AAV ITRs may be derived from any of several AAV serotypes, including but not limited to AAV-1, AAV-2, AAV-3, AAV-4, AAV5, AAV8 or AAV-9.

The expression cassette can also be encompassed in a viral particle. Typically, the expression cassette inserted into an expression vector which is packaged or encapsidated in a viral particle. As used herein, the “viral particle” refers to the packaged or encapsidated viral vector that is capable of binding to the surface and entering inside the host cells. The techniques for isolating viral particles of this invention from host cellular constituents and eventually from other types of viruses (such as helper viruses) which may be present in the host cell, are known to those of skill in the art, and include, for example, centrifugation and affinity chromatography. Typically, the viral particle may be an AAV particle.

“Adeno-associated virus” or “AAV” belong to the Parvoviridae virus family and are of the Dependovirus genus. Wild-type AAVs are low integrative viruses but not lytic and nonpathogenic to humans. They infect a wide variety of mitotic and quiescent cells but are dependent on a helper virus for their replication, such as adenovirus or herpes virus.

As used herein, the term “rAAV” refers to a recombinant AAV-nucleic acid molecule containing some AAV sequences, usually at a minimum the ITRs and some foreign or exogenous (i.e., non-AAV) DNA, such as the NGB nucleic acid sequence of the invention

As used herein, the term “serotype” refers to an AAV which is identified by and distinguished from other AAVs based on capsid protein reactivity with defined antisera. For example, AAV2 serotype 9 (AAV2/9) is used to refer to an AAV composed by the AAV2 Rep regulatory protein and AAV9 cap genes. The virus particle serotype determines its tropism. In the context of the present invention, the AAV2/9 is particularly advantageous due to its strong tropism towards neurons (see for review Huang, et al. Life Sciences 270 (2021): 119142 or Abulimiti, et al. Mechanisms of Ageing and Development 199 (2021): 111549). Therefore, according to a specific embodiment, the neuroglobin-encoding polynucleotide is comprised in an AAV2/9 vector. AAV2/9 vectors allow transducing cells from several tissues, and especially from the central nervous system whose functional integrity is compromised by the progressive and irreversible loss of neurons in patients suffering from neurodegenerative diseases. AAV2/9 vectors are able to naturally bypass the blood-brain-barrier (BBB), thereby allowing for a broad and long-lasting ngb expression after a single intravenous injection.

Typically, the capsid protein of the viral particle may comprise at least one tyrosine residue which is mutated to phenylalanine. For example, the capsid protein may be mutated by substitution of at least three tyrosine residues by phenylalanine residues. Mutation of the capsid proteins modifies viral tropism or increases the transduction efficiency of the rAAV vector and reduces host cell damage. Advantageously, the tyrosine 444 of the capsid is substituted by a phenylalanine residue. Typically, the vector is an AAV-2 Y444F.

In another embodiment, the expression vector may be a lentiviral vector comprising sufficient lentiviral genetic information to allow packaging of an RNA genome, in the presence of packaging components, into a viral particle capable of infecting a host cell (such as the target cells).

As used herein, the term “lentiviral vector” refers to a vector derived from (i.e., sharing nucleotides sequences unique to) a lentivirus. The term “lentiviral vector” also refers to a modified lentivirus having a modified proviral RNA genome which comprises a NGB polynucleotide sequence. According to the invention, the lentiviral vectors derivative from the human immunodeficiency virus (HIV).

Alternatively, one way to deliver the polynucleotide across cell membranes in vivo may involve the direct application of high concentration free or naked polynucleotides (typically mRNA or DNA). By “naked DNA (or RNA)” is meant a DNA (RNA) molecule which has not been previously complexed with other chemical moieties. Naked DNA uptake by animal cells may be increased by administering the cells simultaneously with excipients and the nucleic acid. Such excipients are reagents that enhance or increase penetration of the DNA across cellular membranes and thus delivery to the cells delivery of the therapeutic agent. Various excipients have been described in the art, such as surfactants, e.g. a surfactant selected form the group consisting of Triton X-100, sodium dodecyl sulfate, Tween 20, and Tween 80; bacterial toxins, for instance streptolysin O, cholera toxin, and recombinant modified labile toxin of E coli; and polysaccharides, such as glucose, sucrose, fructose, or maltose, for instance, which act by disrupting the osmotic pressure in the vicinity of the cell membrane.

The invention will now be illustrated by means of the following examples. These examples do not intend to limit the scope of the invention.

References to the methods of treatment by therapy or surgery are to be interpreted as references to compounds of the present invention for use in those methods.

### EXAMPLES

Example 1-Comparison of two routes of AAV2/9 vector administration: local (neurosurgery in cerebellar hemispheres) versus systemic (injection in the vein of the retro-orbital sinus)

### Methods

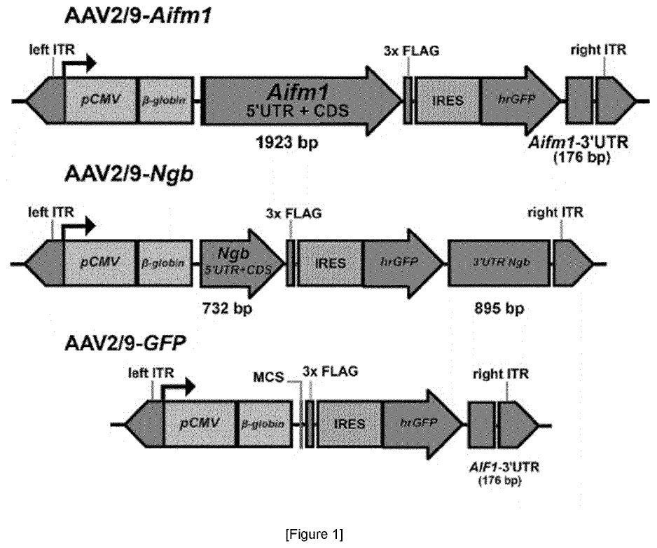

Mice: The Harlequin (Hq) mice originate from the C57BL/6J and B6CBACaAw-J/A-Pdc8Hq/J strains. They harbor a spontaneous mutation in the Aifm1 gene consisting in an ecotropic proviral insertion of 9 kb in the intron 1 of the gene (Jackson Laboratory's strain No. 000501). This insertion leads to a decrease in gene expression with approximately a 90% reduction in Aifm1 messenger RNA and protein levels in Hq mutant mice compared with wild-type control levels (Klein et al. 2002). The Hq mutation is a proviral insertion in the Aifm1 gene, causing about a 90% reduction in Aif expression. The Hq mouse strain exhibits the main features of human neurodegenerative diseases due to RCCI deficiency, such as the degeneration of the cerebellum, retina, optic nerve, thalamic, striatal, and cortical regions. All hemizygous (Hq/Y) male mice used in this study were between F2 and F4 mice bred from founders (Hq/X female mice with wild-type male mice) that had a mixed genetic background and were shipped from The Jackson Laboratory. Only the hemizygous (Hq/Y) mice and their male littermates were evaluated and subjected to gene therapy. The mice were housed in a pathogen-free barrier facility with two to four animals per cage in a temperature-controlled environment, with a 12-hour light/dark cycle and free access to food and water. The animal facility (PHENO-ICMice) is located at the Paris Brain Institute, ICM (see for review the ICM's website). Studies were conducted in accordance with the European Community Council Directive 2010/63/UR, on the protection of animals used for scientific purposes. The scientific project has been authorized regarding the rules on the care and use of animals in research as well as the use of genetically modified organisms (class 2) by the internal scientific committee of the Brain Institute (#P128R) and the French Ministry of Research (#2410). It was also approved by the ethics commissions of the University of Paris and the INSERM (APAFIS #9423-2017032721505008). Production of single-stranded Adeno-Associated Viral Vectors: The AAV2/9-Aifm1 and AAV2/9-Ngb vectors have been obtained as for the serotype 2 (Cwerman-Thibault et al.; Bouaita et al. Brain. 2012; 135 (Pt 1): 35-52; Lechauve et al., 2014). Briefly, the pAAV-IRES-hrGFP vector (Agilent Technologies) was used to insert the murine sequences. The AAV2/9-Ngb vector contains the open reading frame (ORF) (453 bp), the 5′ untranslated region (UTR) (279 bp), and the 3′ UTR (895 bp) of the mouse Ngb mRNA variant 2 (NM_022414.2). The AAV2/9-Aifm1 vector contains the 5′ UTR (87 bp), the entire ORF (1836 bp), and the 176-bp full-length 3′ UTR of the mouse Aifm1 mRNA (NM_012019). The presence in each construction of the full UTR sequences guarantees mRNA stability and translation capacity (Weis et al., Biochim Biophys Acta. 2013; 1833 (2): 260-273; Bae et al., Int J Mol Sci. 2020; 21 (10)). The expression cassettes flanked by the two inverted terminal repeats (ITRs) were packaged in AAV9 shells to ensure a high yield of neuronal cell transduction (Huda et al., Mol Ther Methods Clin Dev. 2014; 1:14032). The AAV2/9-GFP from Agilent contains the 3′UTR from the mouse Aifm1gene and no transgene was inserted in the multi-cloning site (MCS) of the original vector; hence it is used as a negative control. The schematic representation of each vector is illustrated in FIG. 1. Vectors were produced by the Translational Vector Core of the INSERM UMR1089 research unit at Nantes, France.

Stereotactic Neurosurgery: The day of the surgery, the mice undergo a volatile anesthesia (isoflurane 3% for induction and then 2% in a mask). Each mouse is placed in a stereotactic frame and maintained on a heating mat. An incision is performed in the skin of the skull and two small diameter holes are made with a mini-drill (carbon steel burrs 0.5 mm diameter) in the cranial bone on each side of the cerebellum (6 to 6.5 mm caudal from Bregma and 1.75 to 2 mm laterally from the median line; the coordinates were identified with the Atlas of Franklin and Paxinos). The vector stock is diluted in a NaCl 0.09% solution to obtain a final concentration of 5×10⁹or 7×10⁹vector genomes/μL depending of batch titles. Two UL are injected in each location corresponding to right and left cerebellar hemispheres in a depth of 1.1 mm ventral from the dura mater. The infusion rate is 0.35 μL/minute; the vector is administered with a 10 μL-Hamilton neuro-syringe with a 33 Gauge needle. To minimize the reflux of the vector solution, the neuro-syringe remains in place for 3 minutes, and then it is moved up by 0.3 mm every 45 seconds before its complete extraction. Each animal receives approximately 1.0 or 1.4×10¹⁰ Vector Genomes (VG) per hemisphere; since mice at the time of the treatment weighted approximately 25 g the dose used was ~ 9.5×10¹¹per kg. Next, the surgical site is closed with non-absorbable surgical sutures aseptically. Immediately after the procedure, the mouse receives subcutaneously an analgesic (buprenorphine 0.05-0.1 mg/kg) and 400 μL of physiological serum. The analgesic treatment will be given every 8-12 hours for 5 days after the neurosurgery. The protocol of stereotactic neurosurgery was adapted from the publication of Lin and colleagues (Gene. 2015; 571 (1): 81-90). Stereotactic neurosurgery is classified as of moderate severity; thus, all post-operative cares are given to minimize suffering after the intervention and preserve “animal welfare”. The postoperative evaluation (twice a day) was very thorough during one week for an adequate management of the pain caused by the surgery.

### Retro-Orbital Sinus Injection

The different steps are described below and were adapted from two publications of 2011 (Yardeni et al. Lab Anim (NY). 2011; 40 (5): 155-160), and 2021 (Seldeen et al. Mech Ageing Dev. 2019; 180:49-62):
1) The vectors are prepared under Microbiological Safety Station; the injection volume varies between 50 UL and 150 μL depending on the title of the vector;
2) For the administration of the vector an insulin syringe with a 29 Gauge needle is used;
3) Mice are aged between 24-30 days; their body weights oscillate between 12 to 22 g;
4) The final quantity of vector per injection is ~2.6×10¹¹VG which correspond to ~1.53×10¹³VG/kg (approximately 17-fold more than stereotactic surgery);
5) The animal is placed in a plexiglass box in which isoflurane circulates at 3%;
6) When the animal is completely asleep (slowed heart rate, no response to stimuli), it is removed from the box;
7) The mouse is positioned on a heating mat set at 37° C. to maintain the body temperature of the sleeping animal;
8) Next, the animal is slightly restrained and the eyeball is protruded slightly from its socket before inserting the needle in order to avoid damage to the eye;
9) The needle is inserted into the retro-bulbar sinus, at a 45° angle until a mild resistance is felt by the experimenter indicating the needle tip has reached the bony socket;
10) At this point, the required volume of vector solution is softly and smoothly injected;
11) The needle is left in place for 3 to 5 seconds and withdrawn gently to prevent injury to the eye. No leaking of the solution and not bleeding indicate proper injection;
12) A compress is applied with a light pressure on the injected eye for few seconds to halt the possible bleeding;
13) The injection site is examined for swelling or other visible trauma;
14) The mouse is finally returned to its home cage.

Behavior Evaluations of mice: Control and Hq mice were evaluated along with age-matched untreated counterparts to appreciate their cognitive and motor capacities before gene therapy and 6 months after when the stereotactic surgery was chosen as route of vector administration. When retro-orbital injections were performed, the evaluations were executed 1-2 months post-injections and just before euthanasia, i.e., five months after vector administration.

Locomotor skills, muscle tone and coordination were assessed by subjecting mice to: Grip test, Rotarod and Open-field (Seldeen et al., 2019; Deacon, J Vis Exp. 2013; (75): e2609; Kraeuter et al., Methods Mol Biol. 2019; 1916:99-103). The evaluation of learning and memorization abilities was carried out by using The Novel Object Recognition Test (Miedel et al. J Vis Exp. 2017; (123)) and the Y-Maze, forced alternation (Kraeuter et al. Methods Mol Biol. 2019; 1916:105-111). Finally, mouse responses to acute stress or anxiety-provoking situations were estimated after subjecting mice to Open Field (Kraeuter et al, 2019) and Tail Suspension Test (De Sousa et al. Naunyn Schmiedebergs Arch Pharmacol. 2018; 391 (8): 803-817).

Morphological and functional studies of cerebella from treated mice: After euthanasia, mice were subjected to intracardical perfusion with a 4% paraformaldehyde solution, cerebella were harvested to perform immunohistochemistry (cell number estimations, size of the tissue, transduction yields). Tissues were also collected and kept at −80° C. to be used for biochemical studies (Cycle de Krebs, Respiratory chain), or analyses of steady-state levels of mRNAs and proteins by qPCRs and Western blots respectively as performed for retina studies (Cwerman-Thibault et al, 2021). Cerebella from untreated and treated mice were subjected to Transmission Electron Microscopy analyses (Gilbert et al. Elife. 2021; 10) and to Ultra-high Performance Liquid Chromatography coupled to tandem mass spectrometry (UPLC-MS/MS) analyses (Millan et al., Antioxidants (Basel). 2018; 7 (12)).

### Results

The survival rate of animals subjected to local or systemic vector administration:

When stereotactic surgery within cerebellar hemispheres and injection into the retro-orbital sinus were compared regarding the survival rate of mice, it clearly appears that ROI is better tolerated. For instance, Hq mice treated with AAV2/9-GFP, AAV2/9-Aifm1 or AAV2/9-Ngb, via ROI, revealed a percentage of death before the end of the experiment of 28%, 6.7% and 9.5% respectively while after stereotactic surgery it was of 37.4%, 23.9% and 21.6%.

Mortality extent differences between Hq and control mice subjected to stereotactic surgery may be due to the extreme low pain resistance of Hq mice. Hence, the valuable advantage of the ROI protocol was demonstrated; indeed, because it is much less invasive, fewer animals were deceased after vector administration in both control and Hq groups of mice.

### Body Weight Differences in Mice Subjected to Intravenous or Local Delivery of Gene Therapy Vectors:

Body and cerebellar weights were compared in control and Hq mice subjected to local or intravenous administration of AAV2/9-GFP, AAV2/9-Aifm1 or AAV2/9-Ngb. Mice subjected to ROI were euthanized 5 months after the treatment while mice subjected to local administration of the vectors were euthanized 6 months after vector administration. Overall, the treatment of control mice did not change their body or cerebellar weight regardless of the vector administered and the route of administration used. Thus, intravenous administration of AAV2/9-Ngb leading to the systemic overexpression of Aifm1 or Ngb did not have any deleterious consequence on mouse health status when the body and cerebellar weights were considered.

No major differences in body weights were measured for both routes of administration excepted for an increase of 11.5% in Hq mice treated with AAV2/9-Ngb by ROI relative to Hq mice treated with AAV2/9-Ngb via stereotactic surgery (P=0.0047). Moreover, when Hq mice treated with AAV2/9-GFP were compared with Hq mice treated AAV2/9-Ngb, a significant difference was noticed only for intravenous injection, indeed Hq mice which were subjected to ROI with the AAV2/9-Ngb vector were 16% heavier than their counterparts treated with AAV2/9-GFP (P=0.0002).

The gain of weigh, which is expected after birth and until adult age of mice, was evaluated before the treatment at 2 months of age (surgery) or 1 month of age (ROI) and just before euthanasia: 5 months later (ROI) or 6 months later (surgery).

The increase of body weight was only evidenced (39%) in Hq mice when the AAV2/9-Ngb vector was administered by retro-orbital injection (P<0.0001); while for Hq mice subjected to cerebellar administration with AAV2/9-Ngb; there was six month later only a 5% increase in their body weights (P=0.967).

Hence, not only systemic administration of AAV2/9-Ngb did not induce deleterious effects for Hq mouse health, in the contrary taking in account the body weight it can be said that Ngb overexpression was beneficial for the overall development of Hq mice which are known as exhibiting marked growth retardation (Benit et al. PLoS One. 2008; 3 (9): e3208).

Weight and Size Differences of the Cerebellum from Mice Subjected to Intravenous or Local Delivery of Gene Therapy Vectors:

When comparing the weights of cerebella from Hq mice at the time of euthanasia regarding the route of vector administration, it was found that GFP-treated Hq mice had the tissues with the lowest weights compared to the those of mice which overexpressed either Aifm1 or Ngb (data not shown). Aifm1-treated Hq mice displayed cerebella with a weight superior of 52% or 49% relative to GFP-treated Hq mice respectively for local or intravenous injections; the difference was close of the significance for surgery (P=0.083) and was significant when the two groups of mice subjected to retro-orbital sinus injections were compared (P=0.0465).

Interestingly, the weights of cerebella from Ngb-treated Hq mice were incremented by 36% and 53% when compared to those of GFP-treated Hq mice for local and intravenous vector administration respectively. The difference did not reach significance for local administration (P=0.41) but was significant for ROI (P=0.049).

After the evaluation of cerebellar sections subjected to immunohistochemistry, the whole surface of cerebella from Hq mice treated with either one of the vectors was estimated. With this purpose, cerebellar sections were scanned with the NanoZoomer Digital Pathology 2.0 HT scanner and reconstructed digital images from up to 6 independent sections, corresponding to the median zone (vermis) of the tissue were evaluated. The surface of the entire tissue per section was estimated with the ruler tool of the NDP viewer software and was expressed in mm². The mean of the surface in control mice treated with the AAV2/9-GFP vector via ROI for four mice was 7.79±0.25; this number is very similar to the one found in four control mice treated with the AAV2/9-Aifm1 vector via surgery: 7.99±0.35. Obviously, the dimension of cerebellum in Hq mice is significant smaller than the one of control mice; particularly Hq mice treated with GFP independently of the route of administration, since it was observed a ~64% of reduction. The data gathered for Hq mice subjected to gene therapy were compared for the route of administration and the vector injected. An increase of cerebellar area of Hq mice was observed when AAV2/9-Aifm1 (43.7%) or AAV2/9-Ngb (48.5%) were administered via ROI relative to their counterparts injected with AAV2/9-GFP; the difference is significant for AAV2/9-Ngb-treated Hq mice (P=0.042) while this is not the case for AAV2/9-Aifm1 treated Hq mice (P=0.08).

In conclusion, the data obtained suggest that the treatment with the AAV2/9-Ngb, via intravenous injection led to a less severe body weight loss in Hq mice as well as a less pronounced reduction in the weight of their cerebella. This may reflect a better preservation of the general health status of Hq mice when Ngb is overexpressed in different tissues. Moreover, it can be hypothesized that the protection against Purkinje degeneration when AAV2/9-Aifm1 or AAV2/9-Ngb vector is injected into the retro-orbital sinus should increase despite tissue dimensions which remain smaller than those of control mice. Indeed, both the weight and the surface of the cerebellum from Hq subjected to ROI with AAV2/2-Ngb were significantly higher than their counterparts treated with AAV2/2-GFP via ROI.

Morphological Evaluations of Cerebella from Harlequin and Control Mice Subjected to Retro-Orbital Injections:

Control and Hq mice which received, via retro-orbital injection, AAV2/9-Aifm1, AAV2/9-Ngb or AAV2/9-GFP, were subjected to behavioral assessments 5 months later and euthanized when these tests were completed. Cerebella were dissected and sections of 40 μm were obtained using a cryo-microtome. The floating sections were next used for immunohistochemistry with antibodies against GFP, 33-tubulin, Aif or Ngb combined with the antibody against Calbindin D-28k, which is a reliable marker of Purkinje cells (Kim et al. Korean J Physiol Pharmacol. 2009; 13 (5): 373-378; Orduz et al. Front Cell Neurosci. 2014; 8:364). The fluorescence intensity of GFP, Aif or Ngb labeling was high in treated control or Hq mice. The signal corresponding to antibodies which recognized the proteins synthesized from each vector was strong in all the cell layers indicating that an array of neuronal populations within the tissue were efficiently transduced. It arises from these results that the overall cerebellar structure of Aifm1- and Ngb-treated Hq mice is better preserved relative to GFP-treated Hq mice when the vector was delivered by intravenous injections. Reconstructed images for the whole tissues from control and Hq mice subjected to stereotactic surgery were compared to those obtained from their counterparts subjected to intravenous injections. No changes in the overall cerebellar morphology were noticed when control mice were subjected to ROI whatever vector was used. Thus, the delivery of 16-fold more of vectors via an intravenous injection did not lead to deleterious consequences in the cerebellum of these mice. This was also the case for Hq mice; indeed, these mice exhibited a better-preserved morphology of their cerebella. Moreover, cerebella from Aifm1-treated Hq mice and Ngb-treated Hq mice, via ROI, were bigger than those from Hq mice treated via surgery which corroborates the estimation of cerebellar surfaces evaluated earlier. Hence, vector administration via ROI may be more protective against tissue degeneration than surgery.

### Purkinje Cell Number and Connectivity in Harlequin Mice Subjected to Gene Therapy Either Locally or Via Intravenous Injection.

Hereditary ataxias are predominantly due to Purkinje cell degeneration (Robinson et al. Front Neurosci. 2020; 14:707). These cerebellar neurons are especially vulnerable, probably because they are one of the largest neuronal cell types in the brain with a high metabolic activity and extensive dendritic arbors receiving widespread excitatory inputs (Huang et al. Neurosci Lett. 2019; 688:49-57). Noteworthy, Purkinje cells receive more synaptic inputs than any other neuron in the brain, the number of spines on a single human Purkinje cell is as high as 200,000. After emitting collaterals that affect nearby parts of the cortex, their axons travel into the deep cerebellar nuclei, where they make about 1,000 synapses each with several types of nuclear cells, hence they are the sole neurons sending outputs from the cerebellar cortex (Hirano et al. Cerebellum. 2018; 17 (6): 699-700).

For all of these reasons, Purkinje cells and their connections require robust mitochondria for functioning, as demonstrated in Spinocerebellar ataxia 28, due to mutations in the AFG3L2 gene which encodes a mitochondrial metalloprotease involved in the organelle quality control (Almajan et al. J Clin Invest. 2012; 122 (11): 4048-4058). In Hq mice, the depletion of the mitochondrial Aif results in cerebellar degeneration due to granule cell loss and the disappearance of Purkinje cells. Different estimations of cellular loss kinetics and our experience in untreated Hq mice (cellular counts and electronic microscopy) indicate that the degeneration of granule cells in Hq mice begins at the age of 1-2 months; while Purkinje cells disappear 2 months later. By the age of 12-14 months, Hq mice have lost more than 50% of granule cells in the posterior lobe and virtually all Purkinje cells in the lobules VI-VIII (Klein et al.; El Ghouzzi et al. J Neuropathol Exp Neurol. 2007; 66 (9): 838-847; Chung et al. Dis. 2011; 41 (2): 445-457). Hence, Harlequin mutant mice showed severe motor deficits with a markedly altered and unsteady gait as well as diminished muscle tone. These features are easily visible by eye in animals aged 4 months (Preisig et al., Behav Brain Res. 2016; 311:340-353).

Cerebellar sections from Aifm1- and Ngb-treated mice were observed by confocal microscopy after immunolabeling with the antibody against Calbindin and compared with cerebellar sections from GFP-treated control mice and GFP-treated Hq mice. The objective was to strengthen the hypothesis that Purkinje cell degeneration was reduced in Hq mice subjected to AAV2/9-Aifm1 or AAV2/9-Ngb vector administration by ROI relative to stereotactic surgery. Images confirmed that Purkinje cells, in Aifm1- and Ngb-treated Hq cerebella, exhibited a preserved structure and that their dendritic arborizations were more elaborated than the ones displayed by GFP-treated Hq mice. Harlequin mice subjected to an intravenous injection of AAV2/9-GFP vector showed few Purkinje cells, poorly labeled with the anti-calbindin antibody. Conversely, in cerebellum from Hq mice treated with AAV2/9-Aifm1 or AAV2/9-Ngb, Purkinje cells are strongly labeled with the anti-calbindin antibody and several expanded connections were visible from their soma to the molecular cell layer. Additionally, the abondance of Aif or Neuroglobin is high in their soma and dendrites. Control mice treated with AAV2/9-Aifm1 or AAV2/9-Ngb displayed a pattern of labeling similar to their Hq counterparts, except for dendritic arbors of Purkinje cells which were more elaborated in control mice than in Hq mice. However, Purkinje cell connectivity appeared better preserved in Hq cerebella which overexpressed Aifm1 or Ngb relative to GFP-treated Hq mice.

An estimation of: (a) Purkinje cell number in the posterior area of each cerebellum (lobules VI to X); (b) the length of the Purkinje cell layer (mm) within these lobules in order to calculate the cellular density by the normalization of neuronal number against this measure; was then performed. With this purpose in mind, cerebellar sections from the vermis were selected and histochemistry with an antibody against calbindin was carried-out. The number of calbindin-positive cells were counted in the lobules VI to X corresponding to the posterior zone of the tissue.

(1) In Hq mice subjected to local administration of the vectors, the number of Purkinje cells is increased by 72.4% or 84.4% in Hq mice treated with AAV2/9-Aifm1 or AAV2/9-Ngb compared to that obtained for the Hq mice treated with the AAV2/9-GFP vector (P=0.025 or 0.0075 respectively). When Hq mice subjected to ROI were compared, a better and significant preservation of Purkinje cells was observed when Aifm1 or Ngb were overexpressed relative to GFP-treated Hq mice with an 1.8-fold and 2.06-fold increase in the number of Purkinje cells (P=0.0071 and 0.0003 for AAV2/9-Aifm1 or AAV2/9-Ngb respectively). Purkinje cell number in Hq mice subjected to ROI of AAV2/9-Aifm1 or AAV2/9-Ngb was 31% and 40% higher than the one measured in Hq mice treated with the same vectors locally. The differences observed were highly significant (P<0.0001 for both vectors).

(2) When the length of the Purkinje cell layer was compared in Hq mice subjected to surgery, no changes were evidenced. To the contrary, this measure was increased in Hq treated with AAV2/9-Aifm1 or AAV2/9-Ngb relative to Hq treated with AAV2/9-GFP when the intravenous route was used. Indeed, an increase of 42.4% and 29.2% was observed in tissues overexpressing Aifm1 or Ngb relative to GFP-treated Hq mice; these data do reach statistical significance (P=0.0043 for mice treated with either AAV2/9-Aifm1 or AAV2/9-Ngb).

(3) The most striking amelioration noticed was the Purkinje cell density for both routes of vector administration. An increase of 61.5% or 77.4% in Hq mice which overexpressed Aifm1 or Ngb relative to the value measured in GFP-treated Hq mice (P=0.0022 for both vectors) was observed in mice subjected to injection within their cerebella. When Hq mice treated via ROI were compared, a 22% and 52.5% increase was noticed in Hq cerebella which expressed Aifm1 (P=0.823) or Ngb (P=0.017) relative to Hq mice treated with the AAV2/9-GFP vector.

There is no doubt that when Aifm1 or Ngb vectors were administered, via retro-orbital injection, the number of Purkinje cells in the posterior part of the tissue were higher than when the administration was performed by stereotactic surgery in cerebellar hemispheres.

This difference may result from the higher transduction yield of cerebellar neurons when vectors are delivered by intravenous injection instead of a direct injection inside the cerebellum which ultimately should led to a better prevention of Purkinje cell degeneration. Cerebellar injection with AAV2/9-Aifm1 or AAV2/9-Ngb led to 57.6% or 56.7% of transduced Purkinje cells respectively; while 71.1% or 80.3% of Purkinje cells were transduced when AAV2/9-Aifm1 or AAV2/9-Ngb was delivered by ROI.

The protocol of gene therapy through vector injection into the retro-orbital sinus, despite the use of 17 times more of each vector compared to the dose used in mice subjected to surgery, did not result in deleterious effects for the health status of Hq mice. Moreover, improvements in different histochemical parameters evaluated were observed: a) the weight and the surface of the tissue were better preserved; b) cerebellar morphology deterioration was less pronounced; (c) ameliorations were observed in Purkinje cell number and connectivity.

Assessment of Mitochondrial Function in the Cerebellum from Harlequin Mice Subjected to Gene Therapy by Retro-Orbital Sinus Injection

Cerebellar homogenates from mice subjected to ROI and euthanized 5 months later were isolated and subjected to spectrophotometry, as previously described for mouse retinas (Cwerman-Thibault et al. (2021); Bouaita et al. (2012); Lechauve et al. (2014)) for evaluating the enzymatic activities of respiratory chain complexes I and IV as well as of citrate synthase (a component of the Krebs cycle). The analysis following analysis aims at establishing whether energy status restoration could be found after a systemic delivery of the vectors as was demonstrated for stereotactic surgery.

A significant enhancement of Complex I (CI), Complex IV (CIV) and Citrate Synthase (CS) activities in Hq mice injected with the AAV2/9-Ngb vector when compared to the activities measured in control mice treated with the same vector, and this independently of the route used to deliver the vectors was observed. Harlequin mice treated with the AAV2/9-GFP vector exhibited a significant defect of Complex I activity since a diminution of 49% or 44% was observed relative to the activity in GFP-treated control mice when gene therapy was performed by surgery or by intravenous injection (P<0.0001 or P=0.0055). Cerebella from Ngb-treated Hq mice attained 83.3% and 98.2% of the values measured in Ngb-treated controls when the vector was delivered by surgery and ROI (P=0.62 and >0.99 respectively). Complex I activity in Ngb-treated Hq mice was increased of 50.6% or 67.1% when compared to the measurements in cerebella from GFP-treated Hq mice for a local or intravenous administration of the vector. These values were significantly different: P=0.026 and 0.014 for the surgery and intravenous injection respectively.

The activity of Complex IV was also significantly reduced in the cerebella of GFP-treated Hq mice since they corresponded of 58.5% or 51.2% of the values measured in GFP-treated control mice when the vector was delivered via surgery or ROI (P=0.0022 or 0.0013 respectively). When the values for cerebella from Ngb-treated Hq mice were compared to Ngb-treated controls, it was observed that in Hq mice the values attained 92.4% and 110.7% of those measured in control mice when the treatment was performed by surgery or intravenous injection (P=0.92 or >0.99 respectively). Complex IV activity in Ngb-treated Hq mice was increased of 63.5% or 79.0% when compared to cerebella from GFP-treated Hq mice for a local or intravenous administration of the vector. These values were significantly different: P=0.0048 and 0.0008 for surgery and intravenous injection respectively.

The assessment of Citrate Synthase activity confirmed the impairment of bioenergetic status in the cerebellum of Hq mice; indeed, in GFP-treated Hq mouse cerebellar homogenates it was observed a significant decrease of 28.8% and 30.7% in this activity relative to GFP-treated control mice when surgery and ROI routes of vector administration were compared (P=0.0004 and <0.0001 respectively). The diminution observed in Hq mice could be due to a less efficient enzyme or a diminution of the overall mitochondrial content in the tissue since Citrate Synthase activity is considered as an accurate measurement of both mitochondrial content and functionality (Van Bergen et al. Mitochondrion. 2014; 15:24-33). Next, it was observed that Citrate Synthase activity in cerebella from Ngb-treated Hq mice was very similar to those measured in Ngb-treated controls: 107.0% and 113.2% when the treatment was performed by surgery or intravenous injection (P=0.86 and 0.022 for both routes of vector delivery). When the comparison was made between Hq mice treated with AAV2/9-Ngb and Hq mice treated with AAV2/9-GFP, an enhancement of 50.2% and 53.4% was observed for surgery and ROI. Thus, a significant difference exists between these mice: P=0.0005 and <0.0001 for surgery and intravenous injection.

Overall, it may be said from these data that cerebella from Hq mice exhibit a severe defect in CI and CIV activities. The administration of AAV2/9-Ngb vector was beneficial for both complex activities and this independently of the route of administration used for vector delivery. Moreover, the CS activity was also reduced in GFP-treated Hq mice relative to their control counterparts, the administration of AAV2/9-Ngb resulted in an almost complete recovery of the enzymatic activity regardless how the vector was delivered. This is specially the case for Hq mice which received AAV2/9-Ngb via ROI, indeed, a significant difference was calculated between GFP-treated Hq mice and Ngb-treated Hq mice (P=0.022). Hence, Hq mice subjected to ROI with 17-fold more of AAV2/9-Ngb vector (2.6×10¹¹ vector genomes, VG) did not exhibit deleterious effects for the cerebellar bioenergetic status relative to their counterparts which received the vector in cerebellar hemispheres (1.3×10¹⁰ VG).

It is also worth mentioning that when Hq mice were subjected to intravenous injection of AAV2/9-Ngb, the recovery of CI and CIV activities relative to Hq mice treated with AAV2/9-GFP was noticeable higher relative to the direct administration inside the cerebellum as if this protocol led to a better protection of mitochondrial homeostasis.

Evaluations of Cognitive and Motor Performances of Harlequin and Control Mice Subjected to Gene Therapy by Injection into the Retro-Orbital Sinus

The following tests were performed: Grip test (assessment of muscular strength), the tail suspension test (assessment of anxiety-like behavior), Y-Maze, force alteration test (evaluation of spatial reference memory) and the Open Field (measure of total ambulatory activity). The abilities of Ngb-treated Hq mice were considerably improved when compared to those of GFP-treated Hq mice.

Grip test: GFP-treated Hq mice exhibited a reduction of about 50% of their performance relative to GFP-treated control mice, which indicates a significant diminution in their whole-limb muscular strength for both routes of AAV2/9-GFP vector administration (P<0.0001). When the performance of Ngb-treated Hq mice were compared to Ngb-treated controls, an improvement was evidenced for both routes of vector administration since the muscular strength of Ngb-treated Hq mice reached 73.1% and 78.0% relative to Ngb-treated control mice respectively for local surgery and intravenous injection. The difference between GFP-treated Hq mice and Ngb-treated Hq mice was significant for both routes of vector delivery (P<0.0001). Ngb overexpression in the cerebellum of Hq mice, relative to GFP-treated Hq mice results in an improvement of their muscular strength, which is higher for ROI delivery (60.3%) than local injection (43.6%).

Tail suspension test (TST): The assay consists in hanging the mouse by the tail on the edge of a shelf for 6 minutes. The immobility correlates with a “depressive state” and is calculated during the last 4 minutes of the test (Can et al. J Vis Exp. 2012; (59): e3769). GFP-treated Hq mice exhibited an increase of 60.08% and 53.5% in the immobility time relative to GFP-treated controls when vectors were administered locally or via ROI were respectively used for vector delivery). This increase indicated that they experimented an enhanced depressive-like behavior when compared to control mice. In contrast, Ngb-treated Hq mice, responded to the test similarly than Ngb-treated controls (P =0.41 or >0.99 for surgery or ROI vector delivery respectively).

Consequently, the performance of Ngb-treated Hq mice relative to GFP-treated Hq animals were significantly different independently of where the vector was administered (P=0.0018 and 0.0002 for local or ROI respectively). Interestingly, the improvement of the response for Ngb-treated Hq mice relative to GFP-treated Hq animals was higher when mice were subjected to ROI relative to local vector delivery: 47% and 33% respectively.

Y-Maze (forced alternation): This test allows to assess short-term spatial memory (Kraeuter et al. (2019)); GFP-treated Hq mice displayed severe spatial memory perturbations relative to GFP-treated control mice; indeed, their response represented only 23.1% or 36.6% relative to the value measured in GFP-treated controls (P<0.0001 for both local or ROI delivery). The treatment with AAV2/9-Ngb of Hq mice led to a significant increase in the response of these mice relative to GFP-treated Hq (P=0.0081or 0.0009 respectively for local or ROI). In Ngb-treated Hq mice, the capacity of exploring the novel arm reached 79.9% and 93.7% of the value measured in their control counterparts treated with AAV2/9-Ngb by surgery and intravenous injection. Accordingly, there was not significant difference between Ngb-treated Hq mice when either surgery or ROI were used (P=0.64 or >0.99 respectively). Interestingly, the comparison of Ngb-treated Hq mice to their counterparts treated with AAV2/9-GFP evidenced that the spatial memory skills were enhanced after ROI: 2.7-fold relative to 2.4-fold when mice were subjected to local vector injection. Lastly, the short-term spatial memory significantly improved in Hq mice treated with AAV2/9-Ngb when the vector was delivered either via local surgery or intravenous injection and a better response for ROI was observed.

Open field: The open field test is used to evaluate locomotor activity and emotional response (Kraeuter et al. (2019); Seibenhener et al. J Vis Exp. 2015; (96): e52434,54). The analyses of the total ambulatory activity of treated mice allowed to establish differences between both routes of vector administration. Motor function of Hq mice was impaired, indeed, the distance traveled by GFP-treated Hq mice reached only 27.4% or 43.9% of the value obtained from their control counterparts when the vector was delivered either by local surgery or by intravenous injection. The difference between the groups was significant (P<0.0001). The ambulatory activity of Ngb-treated Hq mice subjected to local surgery increased of 75% relative to Ngb-treated control mice (P=0.0095). Besides, Ngb-treated Hq mice, via ROI, exhibited 55.6% more of locomotor activity than GFP-treated Hq mice, and this performance was statistically significant (P=0.0009). Interestingly, the rescue of locomotor activity for Ngb-treated Hq mice relative to Ngb-treated controls was of 60.4% for local vector administration and of 79.2% for vector delivered by ROI (P<0.0001 and 0.49 respectively). Consequently, the difference between Ngb-treated Hq and Ngb-treated controls remained significant for local administration of the vectors (P=0.0095) while for ROI the difference was not more statistically different (P=0.067). Therefore, like for the Y-Maze test, the performance of Hq mice treated with AAV2-Ngb, via ROI, was very similar to the one of their control counterparts. Remarkably, the locomotor activity in Ngb-treated Hq was significantly improved after ROI when compared to local surgery.

### CONCLUSIONS

The ultimate objective of this research project is to develop a gene therapy protocol leading to Ngb overexpression that will have a broad spectrum of action aimed at improving life conditions of patients suffering from an array of neurological handicaps. Not only primary mitochondrial diseases caused by mutations in genes encoding mitochondrial proteins but also brain disorders in which symptoms aggravate consecutive to severe mitochondrial impairment (Zhou et al. Cells. 2018; 7 (12)), such spinocerebellar ataxia (Sullivan et al. J Neurol. 2018; 266 (2): 533-544) and leukodystrophies (Van Der Knaap et al. Lancet Neurol. 2019; 18 (10): 962-972). Indeed, the working hypothesis is that if mitochondrial impairment is not treated in such patients, therapies designed to target the primary genetic defect will be insufficient.

The use of gene therapy, via stereotactic surgery to target cerebellar neurons in Hq mice revealed limitations. Retro-orbital sinus injections (ROIs) provides several improvements: (a) the survival rate of treated Hq mice was significantly increased after ROI relative to cerebellar administration of the vectors via stereotactic surgery; (b) the weight loss of Hq mice subjected to ROI was inferior to the one of Hq mice after stereotactic surgery; (c) a less pronounced reduction of the weight and the dimension of cerebella in Hq mice which received AAV2/2-Ngb by ROI relative to the mice subjected to an injection inside the tissue; (d) the overall morphology of the cerebellum was better preserved when Hq mice were subjected to ROI than when the tissue was directly targeted; (e) the protection of Purkinje cells and their dendritic arborizations appeared enhanced in Hq mice subjected to ROI with the AAV2/9-Ngb vector relative to mice which received the same vector directly into their cerebella. In view of the histochemical studies, it can be concluded that cerebellar integrity in Hq mice was better preserved when gene therapy, with either AAV2/9-Aifm1 or AAV2/9-Ngb, was achieved by intravenous injection instead of by a local delivery.

On the other hand, the data obtained for the bioenergetics status of cerebellar homogenates indicate a significant improvement in the activity of complexes I (CI) and IV (CIV) as well as that of citrate synthase (CS) relative to the values obtained with samples from untreated or GFP-treated Hq mice. The significant improvements of mitochondrial functionality were comparable for the two tested routes of vector delivery: stereotactic surgery in cerebellar hemispheres and vector injection into the veins of the retro-orbital sinus. However, subtle improvements were evidenced in cerebella from Hq mice subjected to AAV2/9-Ngb administration via ROI when compared to injection inside the tissue. For instance, CI and CIV activity improvements were slightly superior when AAV2/9-Ngb was delivered via ROI than when it was injected inside the cerebellum. Moreover, the CS activity was better recovered in cerebella which received AAV2/9-Ngb via ROI relative to a local injection.

Finally, the comparison of the ataxic phenotype, by subjecting mice to behavioral tests, between the two routes of administration needs to be further completed. However, the improvements in terms of muscle strength, locomotor activity, anxiety state and spatial memory of Hq mice treated with the AAV2/2-Ngb vector, via ROI, were real and significant when compared to the performance of untreated or GFP-treated Hq mice. More importantly, Ngb-treated Hq mice, via ROI, performed better than Ngb-treated Hq mice which received the vector inside their cerebella. Thus, the thorough analysis of data gathered from Grip test, Open field, Tail suspension test, and Y-maze-forced alternation test indicates that Hq mice treated with the AAV2/9-Ngb vector by ROI exhibited both an enhanced locomotor capability, a preserved muscular strength and a stronger spatial memory relative to Hq mice which were subjected to a local administration of the vector.

Demonstrating that Ngb-mediated gene therapy is able to lastingly preserve mitochondrial robustness in neurons would represent a pioneering tool to ameliorate life conditions of patients suffering from a large variety of neurological handicaps and be incentive to initiate clinical studies with neuroglobin on neurological diseases such cerebellar ataxias and leukodystrophies (Van Der Knaap et al. (2019); Ghanekar et al. Expert Rev Neurother. 2022; 22 (2): 101-114).

1-10. (canceled)

11. A method for the treatment of a neurological disorder, wherein said method comprises a step of administering intravenously an effective amount of neuroglobin to a patient in need thereof.

12. The method according to claim 11, wherein said neuroglobin is a neuroglobin-encoding polynucleotide.

13. The method according to claim 12, wherein said polynucleotide is comprised in an expression vector.

14. The method according to claim 13, wherein said expression vector is an AAV vector.

15. The method according to claim 14, wherein said expression vector is an AAV2/9 vector.

16. The method according to claim 12, wherein said polynucleotide comprises the neuroglobin gene coding region and a neuroglobin gene 5′UTR and/or 3′UTR.

17. The method according to claim 11, wherein said neurological disorder is associated with a mitochondrial disease associated with respiratory chain complex I (RCCI) deficiency and/or respiratory chain complex III (RCCIII) deficiency.

18. The method according to claim 11, wherein said neurological disorder is hereditary ataxia.

19. The method according to claim 18, wherein said hereditary ataxia is selected from Friedreich ataxia, cerebellar ataxia and spinocerebellar ataxia.

20. The method according to claim 11, wherein the patient receiving the treatment is human.
